(19) United States
(12) Patent Application Publication
(10) Pub. No.: US 2026/0193175 A1
(43) Pub. Date: Jul. 9, 2026

(54) ALKANOLAMINE MULTI-TAILED LIPID, PREPARATION METHOD THEREFOR, AND USE THEREOF

(71) Applicant: South China University of Technology, Guangzhou (CN)

(72) Inventors: Yuan ZHANG, Guangzhou (CN); Zhengqiang FU, Guangzhou (CN)

(73) Assignee: South China University of Technology, Guangzhou (CN)

(21) Appl. No.: 19/560,127

(22) Filed: Mar. 8, 2026

(30) Foreign Application Priority Data
Mar. 24, 2025 (CN) 202510350212.0

(51) Int. Cl. C07C 271/16; A61K 9/51; A61K 47/18; C07C 269/04

(52) U.S. Cl. CPC C07C 271/16; A61K 9/5123; A61K 47/183; C07C 269/04

(57) ABSTRACT

The present disclosure discloses an alkanolamine multi-tailed lipid, a preparation method therefor, and a use thereof. The structural formulas of the lipid such as compound of Formula I, compound of Formula II or their stereoisomers, their tautomers, or their pharmaceutically acceptable salts:

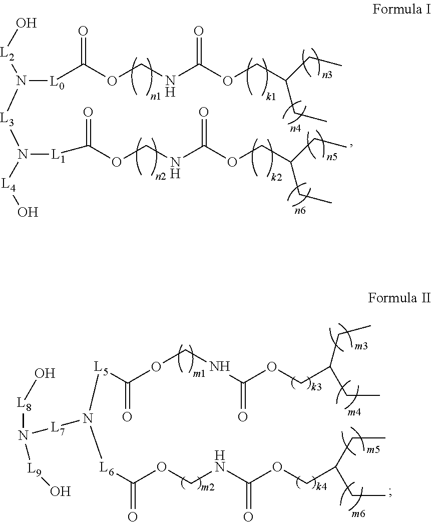

The ionizable lipid compound of the present disclosure maintains nanoparticle stability and delivery efficiency while simplifying LNP composition, offering advantages of high efficacy and low toxicity. Even under neutral conditions, the ionizable lipid compound can still adsorb mRNA through hydrogen bonding and van der Waals interactions. The synthesis method for the diethanolamine multi-tailed ionizable lipid of the present disclosure is straightforward. The ionizable lipid can be prepared on a large scale via a few addition reaction steps, which is convenient for high-throughput material screening.

### TECHNICAL FIELD

The present disclosure pertains to the field of drug delivery technology, particularly to an alkanolamine multi-tailed lipid, a preparation method therefor, and a use thereof.

### BACKGROUND

Ribonucleic acid (RNA) therapy is an emerging technology that employs RNA molecules to treat or prevent diseases. Major modalities of RNA therapy include messenger RNA (mRNA), antisense oligonucleotides (ASOs), and small interfering RNA (siRNA), which achieve therapeutic effects through diverse mechanisms. For instance, mRNA therapy can encode specific proteins to regulate immune responses or to produce therapeutic proteins directly. In recent years, RNA therapy has achieved significant progress in both preclinical and clinical research. This progress, particularly following the successful development of Coronavirus Disease 2019 (COVID-19) mRNA vaccines, has resulted in considerable focus on this technological area. The success of the mRNA vaccines not only demonstrates the potential of RNA therapy in preventing and controlling infectious diseases but also accelerates its application in treating other diseases. Despite advantages such as high specificity, modular development, and relative safety, RNA therapy faces several challenges. RNA molecules are susceptible to in vivo degradation, resulting in a short half-life, which necessitates chemical modifications to enhance their stability. Furthermore, the optimization of delivery systems is a critical factor for the success of RNA therapy, given that effective systems can improve targeting of RNA drugs and reduce immunogenicity. Lipid nanoparticles (LNPs) represent a delivery system that has successfully advanced to clinical research. Notably, the LNP-mRNA vaccines have been deployed in the clinical treatment, representing a significant milestone in the LNP delivery systems.

Compared to conventional cationic liposomes, ionizable lipids exhibit significantly enhanced in vivo stability and transfection efficiency. By remaining electrically neutral when transported in vivo, they contribute to low biological toxicity. The conventional LNP typically includes four components: an ionizable lipid, a phospholipid, cholesterol, and a polyethylene glycol (PEG) lipid. However, the simplification of LNP through component reduction has remained a technical challenge in the delivery field. Three-component LNPs streamline the preparation process of LNPs by reducing their component count while preserving high efficiency and specificity in gene delivery. This design not only enhances delivery performance but also offers new potential for precision medicine. In terms of delivery mechanism, LNPs form a lipid bilayer structure that encapsulates the RNA, facilitating the RNA cross the cell membrane and enter the cytoplasm. Nevertheless, the process of RNA escaping from endosomes into the cytoplasm remains a challenge, requiring further research and technological advancements to enhance delivery efficiency.

### SUMMARY

The present disclosure aims to solve at least one of the above-described technical problems existing in the prior art. Therefore, an objective of the present disclosure is to provide an alkanolamine multi-tailed lipid, a preparation method therefor, and a use thereof.

In order to achieve the above objective, the technical solution adopted by the present disclosure is as follows:

In a first aspect of the present disclosure, an ionizable lipid compound is provided, structural formulas of the ionizable lipid compound such as a compound of Formula I, a compound of Formula II or their stereoisomers, their tautomers, or their pharmaceutically acceptable salts:

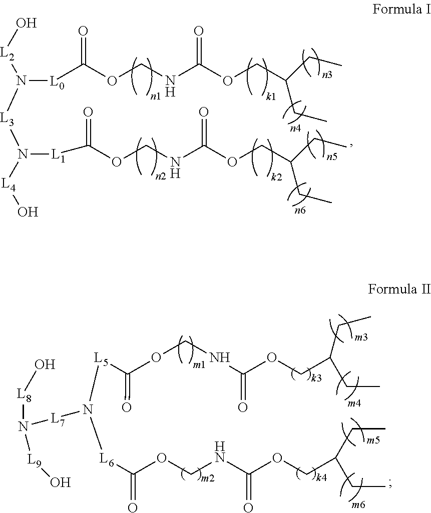

wherein L₀, L₁, L₂, L₃, L₄, L₅, L₆, L₇, L₈, and L₉ are each independently selected from C₁-C₈ alkyl;
n₁, n₂, n₃, n₄, n₅, n₆, m₁, m₂, m₃, m₄, m₅, and m₆ are all natural numbers, and each is independently selected from the natural numbers of 1-20;
k₁, k₂, k₃ and k₄ are all natural numbers, and each is independently selected from the natural numbers of 0-4.

In a second aspect of the present disclosure, a method for preparing the ionizable lipid compound is provided, including the following steps:
reacting a compound of Formula 1 with N,N′-carbonyldiimidazole, followed by reaction with a compound of Formula 3, and subsequently reacting with a C1-C8 alkenyl acyl chloride, to yield an intermediate A; reacting the intermediate A with a compound of Formula 6 to yield the compound of Formula I; and
reacting the intermediate A with a compound of Formula 7 to yield the compound of Formula II.

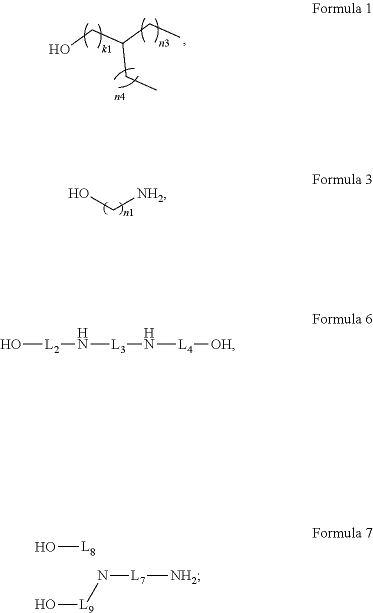

wherein n₁, n₃, n₄, k₁, L₂, L₃, L₄, L₇, L₈, and L₉ are defined as previously described.

In a third aspect of the present disclosure, a lipid nanoparticle is provided, including the ionizable lipid compound.

In a fourth aspect of the present disclosure, a drug composition is provided, including the lipid compound and/or the lipid nanoparticle.

In a fifth aspect of the present disclosure, a use for the lipid compound and the lipid nanoparticle in a manufacture of a drug delivery vehicle.

The beneficial effects of the present disclosure are as follows:

The ionizable lipid compound of the present disclosure is a diethanolamine multi-tailed ionizable lipid, characterized by the ionizable lipid containing multiple tails, and is commonly employed for RNA delivery.

In the present disclosure, the increased cross-sectional area of the tail region in this multi-tailed ionizable lipid facilitates the formation of a more tapered structure with greater endosomal disruptive capability. Furthermore, the headgroup contains a specific diethanolamine group, enabling the construction of a novel three-component mRNA delivery system formulation. This formulation lacks helper lipids and allows for mRNA encapsulation and nanoparticle formation to be completed under neutral pH conditions. The chemical scaffold of the compound contains multiple ester bonds. Following effective release of RNA in vivo, these bonds are susceptible to rapid enzymatic hydrolysis, thereby facilitating in vivo metabolism, clearance and demonstrating biodegradability.

In the present disclosure, the synthesis method for the diethanolamine multi-tailed ionizable lipid is straightforward. The ionizable lipid can be prepared on a large scale via a few addition reaction steps, which is convenient for high-throughput material screening.

The ionizable lipid compound of the present disclosure maintains nanoparticle stability and delivery efficiency while simplifying LNP composition, offering advantages of high efficacy and low toxicity. Even under neutral conditions, the ionizable lipid compound can still adsorb mRNA through hydrogen bonding and van der Waals interactions. This structurally optimized diethanolamine multi-tailed ionizable lipid significantly improves delivery efficiency compared to standard formulations, and the delivery efficiency is better than commercially available lipids.

### BRIEF DESCRIPTION OF THE DRAWINGS

FIG. 1 is a hydrogen spectrum of compound 21-5-C8 of Embodiment 1 of the present disclosure.

FIG. 2 is a hydrogen spectrum of compound 21-5-C8C10 of Embodiment 1 of the present disclosure.

FIG. 3 is a result of relative luciferase activity of Embodiment 2 of the present disclosure.

FIG. 4 is a result of relative luciferase activity in Embodiment 3 of the present disclosure.

FIG. 5 is a result of relative luciferase activity of Embodiment 4 of the present disclosure.

FIG. 6 is a result of relative luciferase activity of Embodiment 4 of the present disclosure.

FIG. 7 is a result of relative luciferase activity of Embodiment 5 of the present disclosure.

FIG. 8 is a result of relative luciferase activity for LNP-self-amplifying RNA (saRNA) delivery in Embodiment 6 of the present disclosure.

FIG. 9 is a result of relative luciferase activity for LNP-nucleoside-modified RNA (modRNA) delivery in Embodiment 6 of the present disclosure.

FIG. 10 is a result of relative luciferase activity for LNP-circular RNA (circRNA) delivery in Embodiment 6 of the present disclosure.

FIG. 11 is an In Vivo Imaging System (IVIS) result on seventh day of in vivo delivery of ionizable lipids in Embodiment 7 of the present disclosure.

FIG. 12 is an IVIS result of in vivo delivery of ionizable lipids in Embodiment 7 of the present disclosure.

FIG. 13 is an IVIS result on seventh day of in vivo delivery of ionizable lipids in Embodiment 8 of the present disclosure; wherein A is 21-5-C8C10:Cholesterol:DMG-PEG2000=30:48:1; B is 21-5-C8C10:Cholesterol:DMG-PEG2000=30:48:2; C is 21-5-C8C10:Cholesterol:DMG-PEG2000=30:48:2.5; D is 21-5-C8C10:Cholesterol:DMG-PEG2000=30:48:5, and ALC-O315 group.

FIG. 14 is an IVIS result of in vivo delivery of ionizable lipids in Embodiment 8 of the present disclosure; wherein A is 21-5-C8C10:Cholesterol:DMG-PEG2000=30:48:1; B is 21-5-C8C10:Cholesterol:DMG-PEG2000=30:48:2; C is 21-5-C8C10:Cholesterol:DMG-PEG2000=30:48:2.5; D is 21-5-C8C10:Cholesterol:DMG-PEG2000=30:48:5, and ALC-O315 group.

### DETAILED DESCRIPTION OF THE EMBODIMENTS

The following is a detailed explanation of the present disclosure through specific embodiments. Unless otherwise specified, the raw materials, reagents, or apparatus used in the embodiment and comparative embodiments may be obtained commercially or produced by existing technical methods. Unless otherwise stated, the experimental or testing methods are conventional methods in the field.

In the first aspect of the present disclosure, the ionizable lipid compound is provided, structural formulas of ionizable lipid compound such as the compound of Formula I, the compound of Formula II or their stereoisomers, their tautomers, or their pharmaceutically acceptable salts:

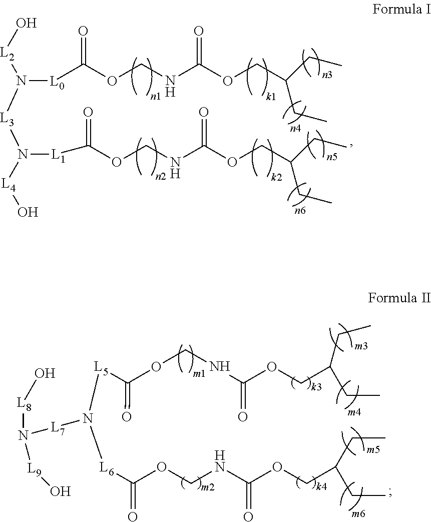

wherein L₀, L₁, L₂, L₃, L₄, L₅, L₆, L₇, L₈, and L₉ are each independently se d from C₁-C₈ alkyl;
n₁, n₂, n₃, n₄, n₅, n₆, m₁, m₂, m₃, m₄, m₅, and m₆ are all natural numbers, and each is independently selected from the natural numbers of 1-20, such as 1, 2, 3, 4, 5, 6, 7, 8, 9, 10, 11, 12, 13, 14, 15, 16, 17, 18, 19, and 20;
k₁, k₂, k₃ and k₄ are all natural numbers, and each is independently selected from the natural numbers of 0-4, such as 1, 2, 3, and 4.

The term “stereoisomer” refers to an isomer characterized by an identical connectivity sequence of atoms but differs in the spatial arrangement of the atoms.

The “tautomer” refers to in a phenomenon where the structure of a compound undergoes equilibrium interconversion between two functional group isomers, and such corresponding isomers are termed tautomers.

The “pharmaceutically acceptable salt” refers to an acid addition salt or a base addition salt. Any compound of the present disclosure in its free base or free acid form can be converted into a corresponding pharmaceutically acceptable salt by reaction with a suitable inorganic or organic acid or base, using methods known to those skilled in the art. And such salts of the compounds of the present disclosure can be formed by conversion to their free bases or acids by standard techniques.

Pharmaceutically acceptable salts of the compounds of the present disclosure include those derived from suitable inorganic and organic acids and bases. Examples of pharmaceutically acceptable, non-toxic acid-addition salts are amino-containing salts formed with inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid, or those formed by other methods used in the art, such as ion exchange. Other pharmaceutically acceptable salts include: adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, tosylate, undecanoate, valerate, and the like. Salts derived from appropriate bases include alkali metal salts, alkaline earth metal salts, and ammonium salts. Representative alkali metal or alkaline earth metal salts include sodium, lithium, potassium, calcium, and magnesium salts, among others. Where appropriate, additional pharmaceutically acceptable salts include non-toxic ammonium, quaternary ammonium, and amine cations formed using counterions such as halide, hydroxide, carboxylate, sulfate, phosphate, nitrate, sulfonate, and arylsulfonate. Further pharmaceutically acceptable salts include those formed by the quaternization of amines, which is performed using appropriate electrophilic reagents (e.g., alkyl halides) to yield quaternized alkylamino salts.

In some embodiments of the present disclosure, the alkyl group is selected from methyl, ethyl, propyl, butyl, pentyl, hexyl, heptyl, or octyl.

In some embodiments of the present disclosure, the compound of Formula I has a structure selected from Formula I-1 or Formula I-2:

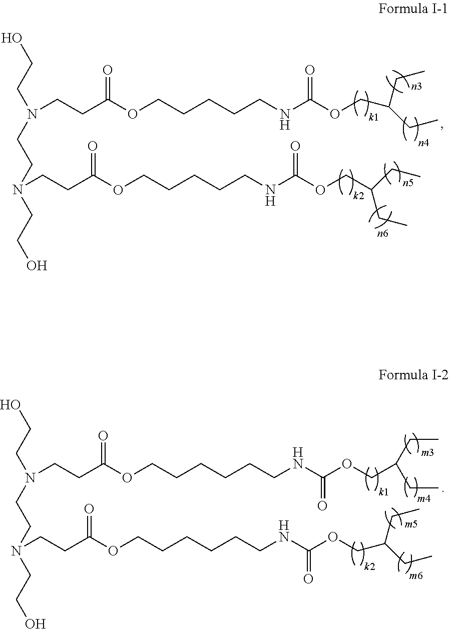

In some embodiments of the present disclosure, the compound of Formula II has a structure selected from Formula II-1 or Formula II-2:

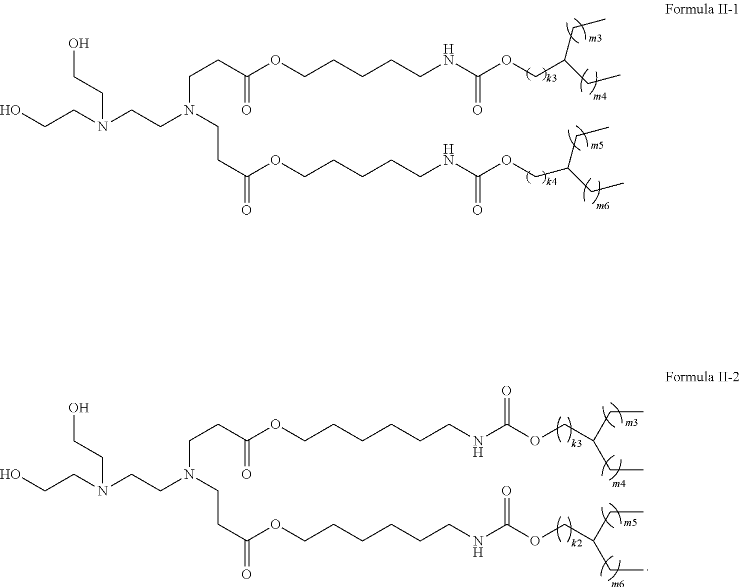

In some embodiments of the present disclosure, the Formula I compound is selected from the following compounds:

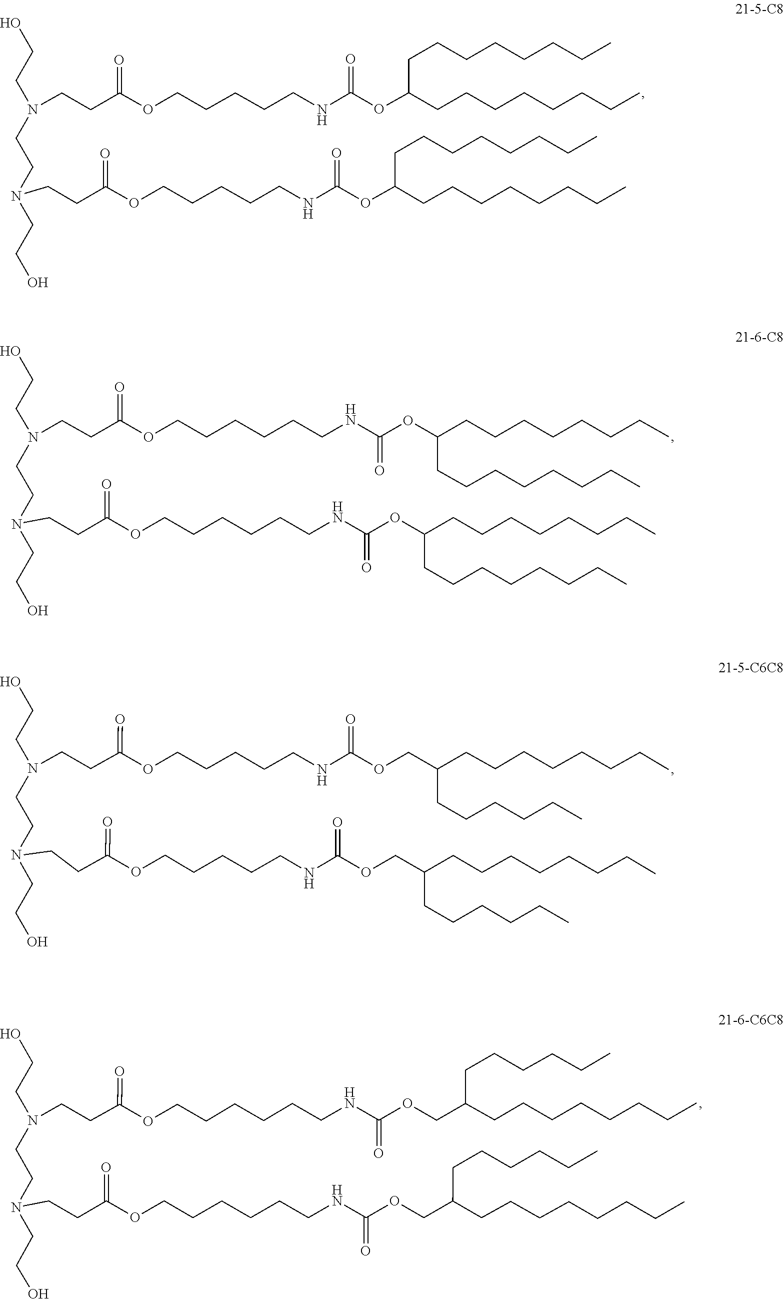 [SMILES: CCCCCCCCC(CCCCCCCC)OC(=O)NCCCCCOC(=O)CCN(CCO)CCN(CCO)CCC(=O)OCCCCCNC(=O)OC(CCCCCCCC)CCCCCCCC ; CCCCCCCCC(CCCCCCCC)OC(=O)NCCCCCCOC(=O)CCN(CCO)CCN(CCO)CCC(=O)OCCCCCCNC(=O)OC(CCCCCCCC)CCCCCCCC ; CCCCCCCCC(CCCCCC)COC(=O)NCCCCCOC(=O)CCN(CCO)CCN(CCO)CCC(=O)OCCCCCNC(=O)OCC(CCCCCC)CCCCCCCC ; CCCCCCCCC(CCCCCC)COC(=O)NCCCCCCOC(=O)CCN(CCO)CCN(CCO)CCC(=O)OCCCCCCNC(=O)OCC(CCCCCC)CCCCCCCC]

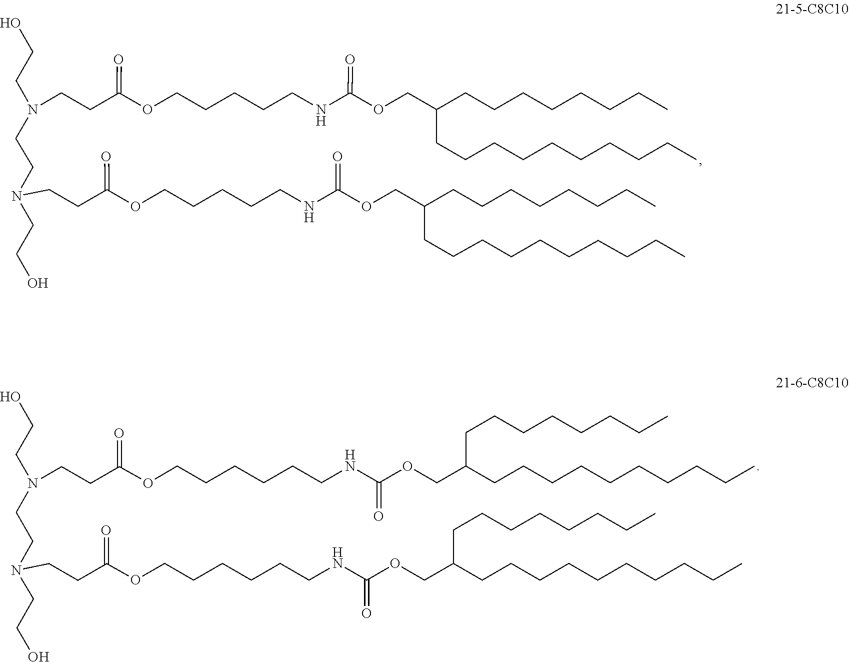 [SMILES: CCCCCCCCCCC(CCCCCCCC)COC(=O)NCCCCCOC(=O)CCN(CCO)CCN(CCO)CCC(=O)OCCCCCNC(=O)OCC(CCCCCCCC)CCCCCCCCCC ; CCCCCCCCCCC(CCCCCCCC)COC(=O)NCCCCCCOC(=O)CCN(CCO)CCN(CCO)CCC(=O)OCCCCCCNC(=O)OCC(CCCCCCCC)CCCCCCCCCC]

In some embodiments of the present disclosure, the compound of Formula II is selected from the following compounds:

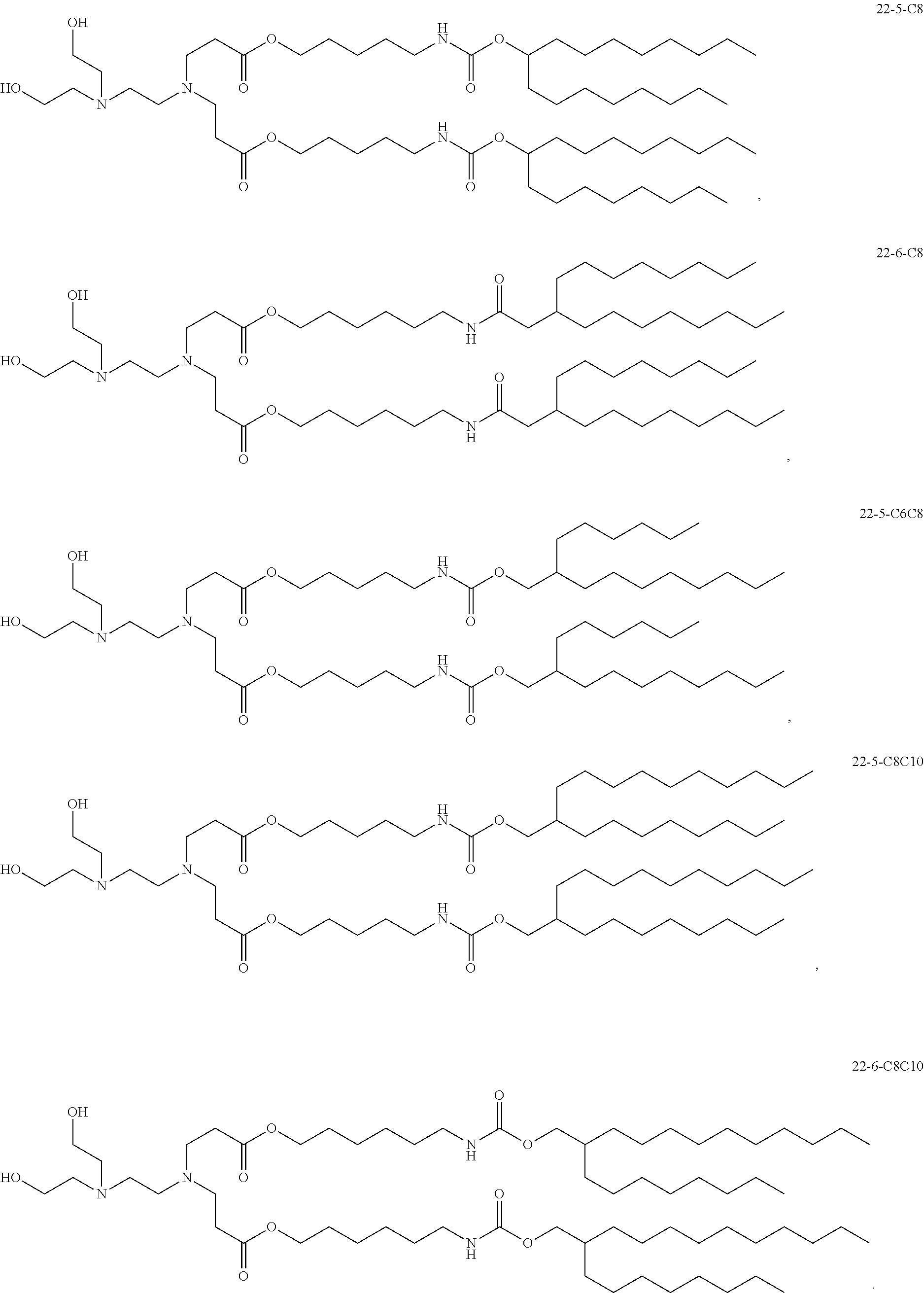 [SMILES: CCCCCCCCC(CCCCCCCC)OC(=O)NCCCCCOC(=O)CCN(CCC(=O)OCCCCCNC(=O)OC(CCCCCCCC)CCCCCCCC)CCN(CCO)CCO ; CCCCCCCCC(CCCCCCCC)CC(=O)NCCCCCCOC(=O)CCN(CCC(=O)OCCCCCCNC(=O)CC(CCCCCCCC)CCCCCCCC)CCN(CCO)CCO ; CCCCCCCCC(CCCCCC)COC(=O)NCCCCCOC(=O)CCN(CCC(=O)OCCCCCNC(=O)OCC(CCCCCC)CCCCCCCC)CCN(CCO)CCO ; CCCCCCCCCCC(CCCCCCCC)COC(=O)NCCCCCOC(=O)CCN(CCC(=O)OCCCCCNC(=O)OCC(CCCCCCCC)CCCCCCCCCC)CCN(CCO)CCO ; CCCCCCCCCCC(CCCCCCCC)COC(=O)NCCCCCCOC(=O)CCN(CCC(=O)OCCCCCCNC(=O)OCC(CCCCCCCC)CCCCCCCCCC)CCN(CCO)CCO]

In the second aspect of the present disclosure, the method for preparing the ionizable lipid compound is provided, including the following steps:
the compound of Formula 1 is reacted with N,N′-carbonyldiimidazole, followed by reaction with the compound of Formula 3, and subsequently is reacted with the C1-C8 alkenyl acyl chloride, to yield the intermediate A; the intermediate A is reacted with the compound of Formula 6 to yield the compound of Formula I; and
the intermediate A is reacted with the compound of Formula 7 to yield the compound of Formula II.

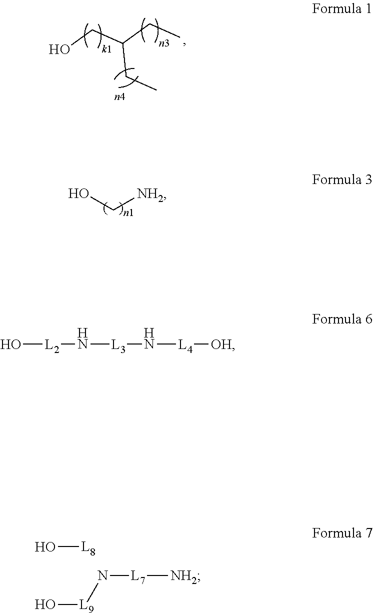

wherein n₁, n₃, n₄, k₁, L₂, L₃, L₄, L₇, L₈, and L₉ are defined as previously described.

Technicians in this field are aware that in the compound of Formula 1, k₁ can also be k₂, k₃, k₄; n₃ can also be n₅, m₃; n₄ can also be n₆, m₆; in the compound of Formula 3, n₁ can also be n₂, m₁, m₂; and they are independent of each other.

In some embodiments of the present disclosure, C1-C8 alkenyl acyl chloride includes acetyl chloride, acryloyl chloride, but-3-enoyl chloride, 4-pentenoyl chloride, 6-chloro-1-hexene, 7-chloro-1-heptene, 8-chloro-1-octene, etc.

In some embodiments of the present disclosure, the intermediate A is reacted with the compound of Formula 6 at 80° C.-100° C. for 12-48 h.

In some embodiments of the present disclosure, the method for preparing the ionizable lipid compound is provided, including the following steps:
the compound of Formula 1 is reacted with N,N′-carbonyldiimidazole at 30° C.-50° C. for 12-48 h, followed by reaction with the compound of Formula 3 at 30° C.-50° C. for 12-48 h, and subsequently is reacted with the C1-C8 alkenyl acyl chloride at 10° C.-30° C. for 6-24 h, to yield the intermediate A; the intermediate A is reacted with the compound of Formula 6 at 80° C.-100° C. for 12-48 h to yield the compound of Formula I; and
the intermediate A is reacted with the compound of Formula 7 at 80° C.-100° C. for 12-48 h to yield the compound of Formula II.

In some embodiments of the present disclosure, when C1-C8 alkenyl acyl chloride are acryloyl chloride, the intermediate A is a compound of Formula 5

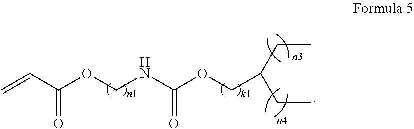

Technicians in this field are aware that in the compound of Formula 5, k₁ can also be k₂, k₃, k₄; n₃ can also be n₅, m₃; n₄ can also be n₆, m₆; n₁ can also be n₂, m₁, m₂; and they are independent of each other.

In the third aspect of the present disclosure, a lipid nanoparticle is provided, including the ionizable lipid compound.

In some embodiments of the present disclosure, the lipid nanoparticle also includes at least one of the structural lipids, auxiliary lipids, polyethylene glycol-modified lipids, and polymers.

In some embodiments of the present disclosure, the ionizable lipid compound and at least one of the structural lipids, auxiliary lipids, and polyethylene glycol-modified lipids are used as drug carriers to deliver drug active ingredients. The lipid nanoparticles of the present disclosure are free of auxiliary lipids and can still effectively deliver drug active ingredients.

In some embodiments of the present disclosure, the molar percentage of the ionizable lipid compound to the structural lipid, the auxiliary lipid, and the polyethylene glycol-modified lipid is in the range of 10-100:0-90:0-90:0-90:0-90, with at least one of the auxiliary lipid, the structural lipid, and the polyethylene glycol-modified lipid being non-zero; such as (20-65):(0-60):(0-10), (20-65):(3-50):(0-60):(0.1-10).

In some embodiments of the present disclosure, the lipid nanoparticles contain 20%-65% ionizable lipid compounds, 0%-40% auxiliary lipids, 20%-60% structural lipids, and 0.1%-10% polyethylene glycol-modified lipids in molar percentage.

In some embodiments of the present disclosure, the lipid nanoparticles contain 35%-49% ionizable lipid compounds, 5%-20% auxiliary lipids, 35%-50% structural lipids, 1%-3% polyethylene glycol-modified lipids, and % refers to the molar percentage.

In some embodiments of the present disclosure, the lipid nanoparticles contain 35%-49% ionizable lipid compounds, 35%-50% structured lipids, 1%-3% polyethylene glycol-modified lipids, and % refers to the molar percentage.

The term “structural lipid” refers to a structure containing a stable composition, including, but not limited to, one or more combinations of sterols and their derivatives and non-sterols and their derivatives.

In some embodiments of the present disclosure, the structural lipids include, but are not limited to: one or more combinations of sterols and their derivatives, non-sterols, sitosterol, ergosterol, cholestaneone, cholestenone, campesterol, stigmasterol, brassicasterol, tomatine, ursolic acid, fecal sterol, α-tocopherol or corticosteroids. Sterol as a preferred cholesterol and its derivatives; unrestricted examples of cholesterol derivatives include: polar analogues such as 5α-cholestanol, 5α-coprostanol, cholesteryl-(2′-hydroxy) ethyl ether, cholesteryl-(4′-hydroxy) butyl ether and 6-ketocholestanol; nonpolar analogues such as 5α-cholestane, cholestenone, 5α-cholestenone and cholesteryl decanoate; and their mixture. In the preferred embodiment, cholesterol derivatives are polar analogues, such as cholesteryl-(4″-hydroxy) butyl ether. This is not an exhaustive list, and the choice of structural lipids is not limited. Any structural lipid can be used in the present disclosure.

In some embodiments of the present disclosure, the structural lipids are one or more combinations of cholesterol, sitosterol, ergosterol, corticosteroids and their derivatives.

In some embodiments of the present disclosure, the structural lipid is cholesterol.

The types of “auxiliary lipids” are not limited, preferably, it is the phospholipid lipid, including but not limited to one or more combinations of phosphatidylcholine, phosphatidylethanolamine, sphingomyelin, ceramide, phosphatidylserine, phosphatidylinositol, phosphatidic acid, phosphatidylglycerol, and dimyristoyl phosphatidylglycerol.

In some embodiments of the present disclosure, the auxiliary lipids may be selected from one or more combinations of 1,2-dioleoyl-sn-glycero-3-phosphoethanolamine (DOPE), 1,2-dioleoyl-sn-glycero-3-phosphocholine (DOPC), 1,2-dilinoleoyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-3-phosphocholine (DUPC), 1-palmitoyl-2-oleoyl-sn-glycero-3-phosphocholine (POPC), 1,2-di-O-octadecenyl-sn-glycero-3-phosphocholine (18:0 diether PC), dimyristoyl phosphoethanolamine (DMPE), distearoyl phosphatidylethanolamine (DSPE), 1,2-dilauroyl-sn-glycero-3-phosphoethanolamine (DLPE), dipalmitoylphosphatidylethanolamine (DPPE), 1-oleoyl-2-cholestyl-succinyl-sn-glycero-3-phosphocholine (OChemsPC), 1-O-hexadecyl-sn-glycero-3-phosphocholine, 1,2-dilinolenoyl-sn-glycero-3-phosphocholine, 1,2-diarachidonoyl-sn-glycero-3-phosphocholine, 1,2-docosahexanoyl-sn-glycero-3-phosphocholine, 1,2-diphytanoyl-sn-glycero-3-phosphoethanolamine, 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-docosahexanoyl-sn-glycero-3-phosphoethanolamine, 1,2-dioleoyl-sn-glycero-3-phospho-rac-(1-glycerol) sodium salt (DOPG), diacetyl phosphatidylethanolamine (DEPE), stearoyl-phosphatidylethanolamine (SOPE), lysophosphatidylcholine and sphingomyelin.

In some embodiments of the present disclosure, phosphatidylcholine is one or more combinations of DSPC, DPPC, DMPC, DOPC, and POPC.

In some embodiments of the present disclosure, the auxiliary lipid is phosphatidylcholine, specifically DSPC.

In some embodiments of the present disclosure, the auxiliary lipid is phosphatidylethanolamine, specifically DOPE.

In some embodiments of the present disclosure, the auxiliary lipids are selected from one or more combinations of DOTAP (1,2-dioleoyl-3-trimethylammonium-propane chloride), DODAP (1,2-dioleoyl-3-dimethylammonium-propane), 18:1PA (1,2-DI (cis-9-octadecenoyl)-SN-glycero 3-sodium phosphate), HS15 (polyethylene glycol (15)-hydroxy stearate), GL67 (N4-arginine cholesteryl carbamate).

The “polyethylene glycol-modified lipid” in the present disclosure typically refers to the conjugate formed by linking polyethylene glycol (PEG) and a lipid molecule via a chemical bond. It includes, but is not limited to, PEG-modified phospholipids and derived lipids, exemplary examples such as one or more combinations of PEG-modified phosphatidylethanolamine, PEG-modified phosphatidic acid, PEG-modified ceramide, PEG-modified dialkylamine, PEG-modified diacylglycerol, PEG-modified dialkylglycerol, and methoxy polyethylene glycol ditetradecylacetamide.

In some embodiments of the present disclosure, the PEG-modified lipid includes, but is not limited to, one or more combinations of PEG-C-DMG, PEG-C-DOMG, PEG-DLPE, PEG-DMPE, PEG-DPPE, PEG-DOPE, PEG-DPPC, PEG-distearoyl phosphatidylethanolamine (PEG-DSPE), PEG-DS, Chol (cholesterol)-PEG, 1,2-dimyristoyl-rac-glycero-3-methoxy polyethylene glycol (PEG-DMG), PEG-S-DMG, polyethylene glycol phosphatidylethanolamine, polyethylene glycol ceramide, polyethylene glycol dimethylacrylate (PEG-DMA), PEG distearyl glycerol, PEG dipalmitoleoyl, PEG dioleoyl, PEG-based distearyl, PEG-based diacylglycamide, PEG dipalmitoylphosphatidylethanolamine, PEG-based phosphatidylethanol, PEG-based phosphatidyl ethylene glycol cardamoyloxypropyl-3-amine, PEG-based oxypropyl alcohol amine, 1,2-distearoyloxypropyl-3-amine-N-[methoxy(polyethylene glycol)](PEG-DSA), methoxypolyethylene glycol laurate, and methoxy polyethylene glycol ditetradecylacetamide (ALC0159).

In some embodiments of the present disclosure, the polyethylene glycol-modified lipid is PEG-DMG.

In some embodiments of the present disclosure, the weight-average molecular weight of polyethylene glycol in the polyethylene glycol-modified lipid is 1000-10000, for example, 1000-2000, 2000-4000, 4000-6000, 6000-8000, and 8000-10000, the preferred value is 2,000.

The type of “polymer” is not limited. Polymers may include, but are not limited to, amphiphilic block copolymers, the amphiphilic block copolymers are block copolymers composed of hydrophobic polymers and hydrophilic compounds, including but not limited to polylactic acid (PLA), poly(lactic-co-glycolic acid) (PLGA), poly(lactic-co-glycolic acid) (PLCG), polycaprolactone (PCL), poly(ortho esters), polyanhydride (PAH), polyphosphazene, poly(β-polyaminoester) (PBAE), poly(α-hydroxy acids), lactide/ethylene glycol copolymer (PLGA or PLG) (which includes lactide/glycolide copolymer, D-lactide/glycolide copolymer, L-lactide/glycolide copolymer and D, L-lactide/glycolide copolymer), polyglycolide (PGA), poly(ortho esters)(POE), linear or branched polyethylene glycol (PEG), conjugates of poly(α-hydroxy acids), polyaspirins, polyphosphazenes, D-lactide, D,L-lactide-caprolactone, D,L-lactide-glycolide-caprolactone, dextran, vinylpyrrolidone, polyvinyl alcohol (PVA), methacrylate, poly(N-isopropylacrylamide), SAB (sucrose acetate isobutyrate) hydroxypropyl cellulose, hydroxypropyl methylcellulose, hydroxyethyl methylcellulose, carboxymethyl cellulose or its salts, carbopol, poly(hydroxyethyl methacrylate), poly(methoxyethyl methacrylate), poly(methoxyethoxy-ethyl methacrylate), poly(methyl methacrylate) (PMMA), methyl methacrylate (MMA), PVA-g-PLGA, PEGT-PBT copolymer, PEO-PPO-PEO (pluronics), PEO-PPO-PAA copolymer, PLGA-PEO-PLGA, PEG-PLGA, PLA-PLGA, PEG-PLA, PEG-PCL, Poloxamer 407, PEG-PLGA-PEG triblock copolymer, PEG-PLA-PEG triblock copolymer, PEG-PCL-PEG triblock copolymer, or block copolymers thereof with polyethylene glycol (PEG), or a combination of one or more of the aforementioned polymers or copolymers.

In the fourth aspect of the present disclosure, the drug composition is provided, including the lipid compound and/or the lipid nanoparticle.

In some embodiments of the present disclosure, the drug composition also includes drug active ingredients and/or pharmaceutically acceptable excipients.

In some embodiments of the present disclosure, the drug active ingredient includes at least one of nucleic acid, small molecule, and protein drugs.

In some embodiments of the present disclosure, the “nucleic acid” may be a nucleotide polymer of any length, including but not limited to, one or a combination of single-stranded DNA, double-stranded DNA, plasmid DNA, short isomers, mRNA, tRNA, rRNA, long-chain non-coding RNA (lncRNA), small non-coding RNA (miRNA and siRNA), telomerase RNA, small molecule RNA (snRNA and scRNA), circular RNA (circRNA), synthetic miRNA (miRNA mimics, miRNA agomir, and miRNA antagomir), antisense oligonucleotide (ASO), ribozyme, asymmetric interference RNA (aiRNA), Dicer-substrate RNA (dsRNA), small hairpin RNA (shRNA), guide RNA (gRNA), small guide RNA (sgRNA), locked nucleic acid (LNA), peptide nucleic acid (PNA), morpholino antisense oligonucleotide, morpholino oligonucleotide or bio-customized oligonucleotide.

In some embodiments of the present disclosure, the nucleic acid is mRNA. The mRNA is a class of single-stranded RNA transcribed from a strand of DNA as a template, carrying genetic information that can guide protein synthesis. mRNA may be monocistronic mRNA or multicistronic mRNA.

In some embodiments of the present disclosure, the “small molecule” refers to a compound that is not a protein or nucleic acid molecule. Such small molecules can act as therapeutic and/or prophylactic agents, and include, for example, antibiotics, anti-inflammatory agents, anti-cancer drugs, antiviral drugs, immunosuppressants, analgesics, antifungal agents, antiparasitic agents, anticonvulsants, antidepressants, anti-anxiety drugs, antipsychotics, etc.

In some embodiments of the present disclosure, the protein drugs include cell colony-stimulating factor, interleukin, lymphotoxin, interferon protein, tumor necrosis factor, antibody, and protein antigen.

The drug composition of the present disclosure further contains pharmaceutically acceptable excipients. Typically, these compositions are formulated in non-toxic, inert, and pharmaceutically acceptable aqueous carrier media, with a pH generally in the range of approximately 4-8, preferably approximately 5-8, or approximately 5-7.5, although the pH may vary depending on the specific compound formulated and the condition to be treated. The prepared drug compositions may be administered via conventional routes, including, but not limited to: intravenous injection, intravenous infusion, subcutaneous injection, local injection, intramuscular injection, intratumoral injection, intraperitoneal injection (e.g., intraperitoneal administration), intracranial injection, intracavitary injection, inhalation drug delivery, and implantable drug delivery.

The “pharmaceutically acceptable” in the present disclosure means that when drugs are given to animals or humans appropriately, they do not produce adverse, allergic or other adverse reactions.

The “pharmaceutically acceptable excipients” should be compatible with the active ingredients, that is, they can be combined without substantially diminishing the drug's efficacy under normal conditions. Specific examples of some substances that may be used as pharmaceutically acceptable excipients may be carbohydrates, such as glucose, mannitol, sucrose, lactose, trehalose, maltose, etc.; starch, such as corn starch and potato starch; cellulose and its derivatives, such as sodium methyl cellulose, ethyl cellulose and methyl cellulose; xihuangzhijiao powder; malt; gelatin; talcum; solid lubricants, such as stearic acid and magnesium stearate; calcium sulfate; vegetable oil, such as peanut oil, cottonseed oil, sesame oil, olive oil, corn oil and cocoa oil; alcohols, such as ethanol, propylene glycol, glycerol, sorbitol, mannitol and polyethylene glycol; alginate; emulsifiers, such as Tween; wetting agent, such as sodium lauryl sulfate; surfactant; freeze-dried protective agent; coloring agent; flavoring agent; tabletting agent; stabilizer; diluent; excipient; antioxidant; preservatives; anhydrous raw water; isotonic salt solution; buffer solution, etc., and their combinations. These substances are incorporated as needed to enhance formulation stability, increase activity or bioavailability, or to provide an acceptable taste or odor in the case of oral dosage forms.

The present disclosure, the drug composition may be formulated into inhalation aerosol preparations (e.g., dry powder formulations, aerosols, and inhalable aerosol droplets), implantable gel formulations, and microneedle formulations. They may also be prepared for injection, such as normal saline or aqueous solutions containing glucose and other adjuvants, using conventional methods. Drug compositions such as injections and solutions should be manufactured under aseptic conditions. The dosage of the active ingredient is a therapeutically effective amount, for example, ranging from about 10 μg/kg of body weight to about 50 mg/kg of body weight per day.

In some embodiments of the present disclosure, the drug composition contains lipid nanoparticles (LNPs). The average particle size of the lipid nanoparticles is 30-150 nm, which can be 60-120 nm, 60-70 nm, 70-80 nm, 80-90 nm, 90-100 nm, 100-110 nm, 110-120 nm, or 60-80 nm, 80-100 nm, 100-120 nm.

In some embodiments of the present disclosure, the nitrogen-to-phosphorus (N/P) ratio of the protonated amino group of the ionizable lipid compound in the drug composition to the nucleic acid drug is 1-100:1, such as 1-60:1, 1-50:1, 5-30:1.

In some embodiments of the present disclosure, the drug composition contains nucleic acid lipid nanoparticles, and the encapsulation efficiency of the nucleic acid in the nano-lipid particles is greater than 80%, which may be 80%-85%, 85%-90%, 90%-95%, 95%-97%, 97%-99% or more than 99%.

### Embodiment 1

In this embodiment, an alcohol amine multi-tailed lipid is prepared. The specific process is as follows:

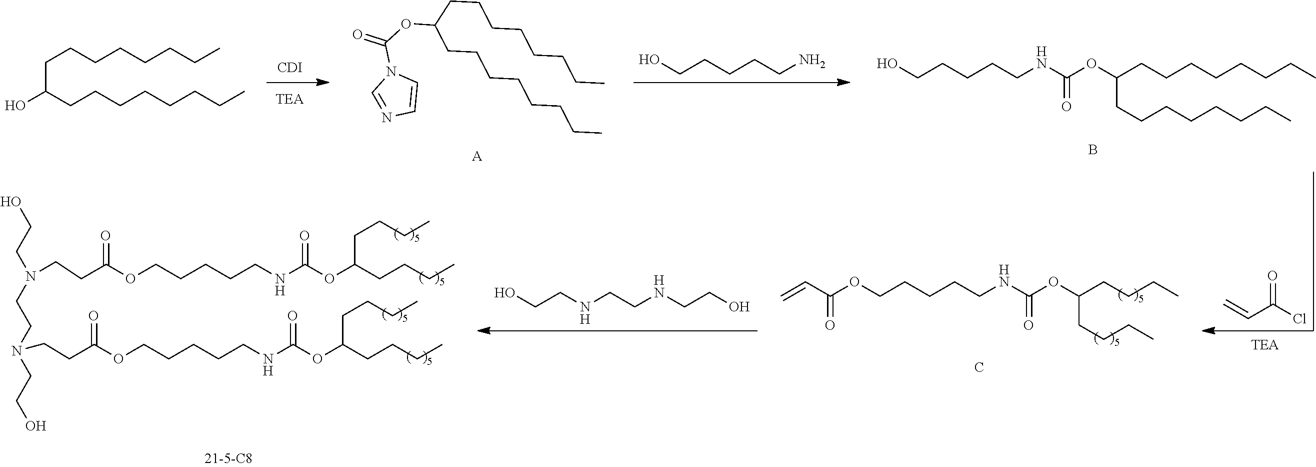

S1: Synthesis of Compound A: 10 mmol 9-heptadecanol, 30 mmol N,N′-carbonyldiimidazole, 20 mmol triethylamine (TEA), 30 mL dichloromethane (DCM) and magnetons are added into a 100 mL reaction tube in turn. The reaction tube is placed in a heating mantle and reacted at 40° C. for 24 h. The reaction progress is monitored by thin layer chromatography (TLC) until completion. The reaction mixture is transferred to a separatory funnel, extracted with DCM (2×100 mL) and saturated brine (2×100 mL), and washed with 1 M hydrochloric acid (HCl) (2×20 mL). The organic layer is collected and dried with anhydrous magnesium sulfate and filtered, and the solvent is removed using a rotary evaporator. The resulting product A is used directly in the subsequent step without further purification.

S2: Synthesis of Compound B: 10 mmol intermediate A, 20 mmol 5-amino-1-pentanol and 30 mL dichloromethane are added into a 100 mL reaction tube containing magnetons in turn. The reaction tube is placed in a heating mantle and reacted at 40° C. for 24 h. The reaction progress is monitored by TLC until completion. The reaction mixture is extracted with dichloromethane and saturated brine. The organic layer is collected and dried with anhydrous magnesium sulfate and filtered, and the solvent is removed using a rotary evaporator. The product is purified by the TCL.

S3: Synthesis of Compound C: 10 mmol intermediate product B, 15 mmol triethylamine and 30 mL dichloromethane are added into a three-necked round-bottom flask containing magnetons in turn. The three-necked round-bottom flask is pre-cooled in an ice bath for 30 min. Then, the solution of 13 mmol acryloyl chloride (pre-mixed in 10 mL dichloromethane) is added dropwise via a pressure-equalizing dropping funnel. After the addition of acryloyl chloride is complete, the ice bath is removed, and the reaction is allowed to proceed at room temperature overnight. The reaction mixture is diluted with DCM (30 mL) and washed with 1 M HCl (50 mL). The organic layer is collected and dried with anhydrous magnesium sulfate and filtered, and the solvent is removed using a rotary evaporator. The product is purified by the TCL.

S4: Synthesis of Compound 21-5-C8: 50 mg N,N′-bis(2-hydroxyethyl) ethylenediamine is added into a 5 mL reaction flask containing magnetons (the cap is lined with tetrafluoroethylene), followed by 2.2 chemical equivalents of intermediate C. The mixture is reacted at 90° C. for 24 h. After the reaction is complete, the product is purified by TCL (dichloromethane:methanol=20:1) to yield ionizable lipid 21-5-C8.

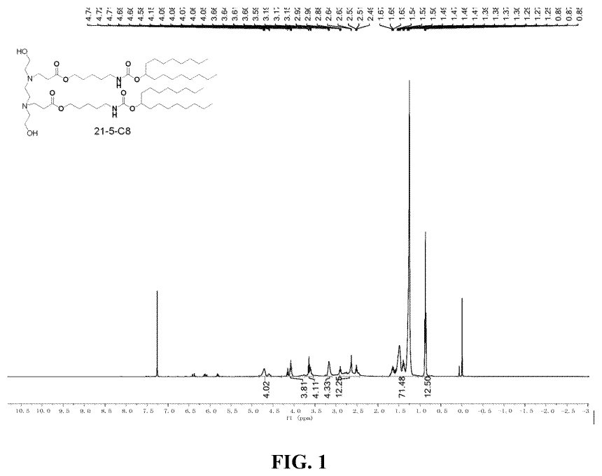

The hydrogen spectrum of the product is shown in FIG. 1, and the hydrogen spectrum data are as follows:

¹H NMR (400 MHz, CDCl₃): 4.74-4.58 (m, 4H), 4.15-4.05 (m, 4H), 3.66-3.59 (m, 4H), 3.19-3.15 (m, 4H), 2.92-2.49 (m, 12H), 1.62-1.25 (m, 72H), 0.87 (t, J=8.0 Hz, 12H).

The difference between the synthesis of compound 21-5-C8C10 and the above steps is that the branched tail is replaced by 2-octyldodecanol in S1, and the other steps are the same, that is, the ionizable lipid 21-5-C8C10 is obtained.

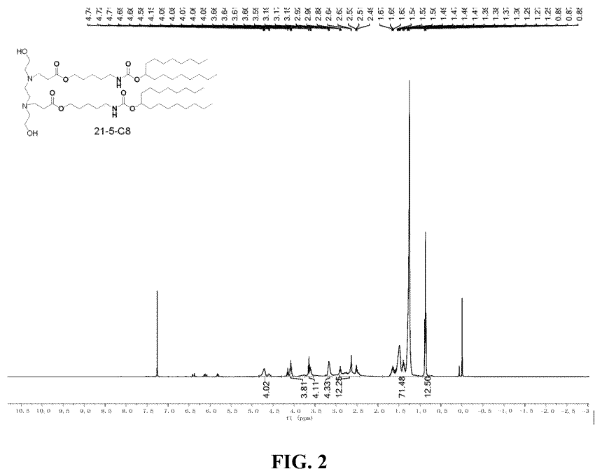

The hydrogen spectrum of the obtained product is shown in FIG. 2, and the hydrogen spectrum data are as follows:

¹H NMR (400 MHz, CDCl₃):4.81-4.66 (m, 2H), 4.15-3.93 (m, 8H), 3.66-3.60 (m, 4H), 3.19-3.14 (m, 4H), 2.90-2.48 (m, 12H), 1.67-1.25 (m, 84H), 0.87 (t, J=8.0 Hz, 12H).

By substituting the corresponding starting materials according to the foregoing method, the following compounds are obtained: 21-6-C8, 21-5-C6C8, 21-6-C6C8, 21-6-C8C10, 22-5-C8, 22-6-C8, 22-5-C6C8, 22-5-C8C10, and 22-6-C8C10. If N,N′-bis(2-hydroxyethyl) ethylenediamine (CAS No.: 4439-20-7) in step S4 is replaced with N,N-bis(2-hydroxyethyl) ethylenediamine (CAS No.: 3197-06-6) while keeping all other steps unchanged, the ionizable lipid 22-6-C8 is obtained.

### Embodiment 2

In this embodiment, an ionizable lipid is used to deliver mRNA, and the specific process is as follows:

The efficiency of ionizable lipid delivery of self-replicating mRNA encoding firefly luciferase (Luc) (saRNA-Luc) is verified in 293T cell lines. Ionizable lipids 21-5-C8, 22-5-C8, 21-6-C8, 22-6-C8, 21-5-C6C8, 22-5-C6C8, 21-6-C6C8, 21-6-C6C8, 21-5-C8C10, 22-5-C8C10, 21-6-C8C10, 21-6-C8C10 and commercial material ALC-0315 are used to deliver saRNA in cells, respectively.

Specific steps are as follows:

### 1. Cell Culture

One day prior to the experiment, the cultured 293T cells are seeded in a 96-well cell culture plate (at a density of 50%). The cell transfection experiments are performed when the cell density reaches approximately 70-80%.

### 2. Preparation of Lipid Nanoparticles LNP-saRNA-Luc for Cell Transfection

Ionizable lipids 21-5-C8, 22-5-C8, 21-6-C8, 22-6-C8, 21-5-C6C8, 22-5-C6C8, 21-6-C6C8, 21-6-C6C8, 21-5-C8C10, 22-5-C8C10, 21-6-C8C10, 21-6-C8C10 are dissolved with distearyl phosphatidylcholine (DSPC), cholesterol, and dimyristoyl glycerol-polyethylene glycol 2000 (PEG-DMG2000) in anhydrous ethanol at specified concentrations. The components are uniformly mixed at a molar ratio of ionizable lipid:Cholesterol:DSPC:PEG-DMG2000=40:48:10:2. Concurrently, 150 ng saRNA-Luc is dissolved in sodium acetate buffer (the volume of sodium acetate buffer is twice the total lipid mixture volume, pH 5.2). Then, the saRNA buffer solution is rapidly mixed with the lipid mixture and incubated at room temperature for 20 min to allow assembly into stable LNPs. The LNP formulation is diluted with two volumes of sterile PBS and added to a 96-well cell culture plate for transfection. The optimal N/P ratio of ionizable lipid to saRNA is 18:1, which represents the molar ratio between the protonated amino groups of the lipid and the phosphate groups on the saRNA.

Positive control group: commercial lipid ALC-0315 assemblies LNPs according to its public preparation method.

Negative control group: 293T cells are cultured normally without transfection.

### 3. Cell Transfection Efficiency Analysis

After 36 hours of cell transfection, the medium in the 96-well cell culture plate is aspirated. A cell lysis buffer is added to lyse the cells on ice for 25 min. Following centrifugation, the supernatant is collected and transferred to a white 96-well detecting plate. Then the firefly luciferase substrate is added, and the firefly luciferase activity (chemiluminescence) is measured using a microplate reader.

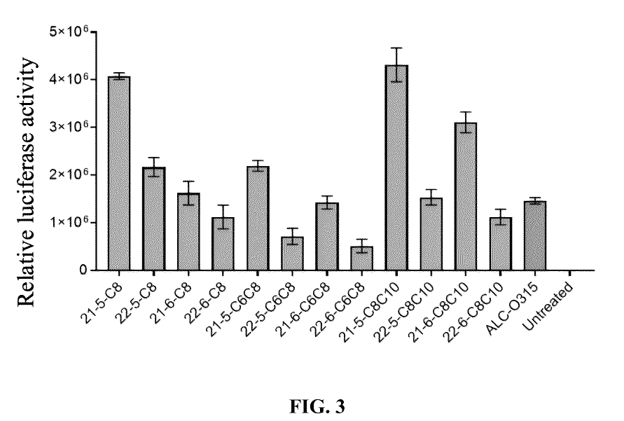

The result of relative luciferase activity is shown in FIG. 3. The result shows that the ionizable lipids synthesized according to the present disclosure significantly enhance the transfection efficiency of self-replicating mRNA. Specifically, when the ionizable lipid headgroup is 21, which exhibits the highest RNA expression efficiency. Representative lipids 21-5-C8 and 21-5-C8C10 show transfection efficiency approximately three times greater than that of the commercial lipid ALC-0315, indicating that the overall chemical architecture of the ionizable lipids designed by the present disclosure is reasonable and efficient.

### Embodiment 3

In this embodiment, an ionizable lipid is used to deliver mRNA, and the specific process is as follows:

The specific operation of the experiment is based on embodiment 2, the ionizable lipid 21-5-C8C10 is used. In the four-component LNP formulation, the molar percentage of distearyl phosphatidylcholine (DSPC) is varied at 20, 15, 10, 5, and 0, respectively, while keeping the other components unchanged.

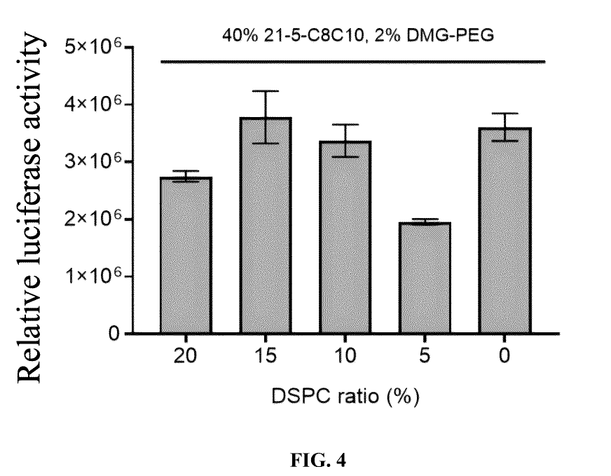

The result of relative luciferase activity is shown in FIG. 4. The results show that the content of DSPC will greatly affect the efficiency of RNA delivery. When the DSPC of LNP is 0%, the ionizable lipid can still efficiently deliver self-replicating mRNA, indicating that the ionizable lipid compound, that is diethanolamine lipid, is capable of efficiently delivering self-replicating mRNA in a three-component system.

### Embodiment 4

In this embodiment, an ionizable lipid is used to deliver mRNA, and the specific process was as follows:

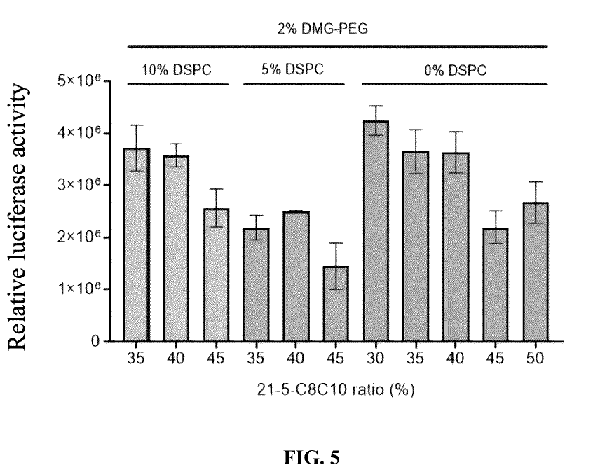

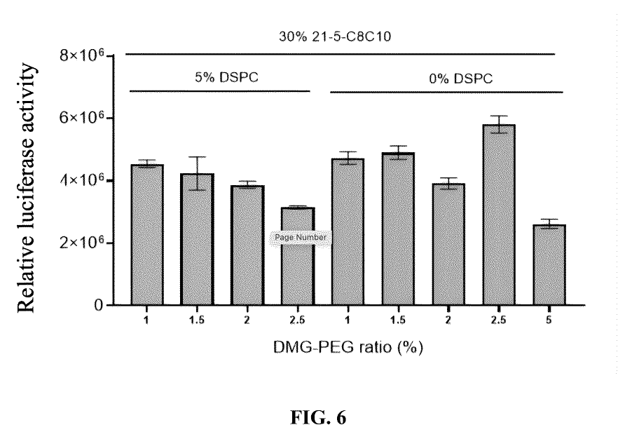

The specific operation of the experiment is based on embodiment 2, the ionizable lipid 21-5-C8C10 is used. In the four-component LNP formulation, firstly, the ratios of DMG-PEG2000 and cholesterol are kept unchanged, while the ratios of 21-5-C8C10 (30%, 35%, 40%, 45%, 50%) and DSPC (0%, 5%, 10%) are optimized (as shown in FIG. 5). Subsequently, with the ratios of 21-5-C8C10 and cholesterol kept unchanged, the ratios of DSPC (0%, 5%) and DMG-PEG2000 (1%, 1.5%, 2%, 2.5%, 5%) are optimized (as shown in FIG. 6).

The results of relative luciferase activity are shown in FIG. 5 and FIG. 6. The molar ratio between the components of LNP also impacts the efficiency of RNA delivery to a certain extent. Ionizable lipid:Cholesterol:DMG-PEG2000=30:48:2.5 (equal to 37.3:59.6:3.1) is the optimal ratio.

### Embodiment 5

In this embodiment, an ionizable lipid is used to deliver mRNA, and the specific process was as follows:

The specific operation of the experiment is based on embodiment 2, the ionizable lipid 21-5-C8C10 is used. The buffer used to prepare the nanoparticles is changed to “150 ng of saRNA-Luc is dissolved in sodium acetate buffer (the sodium acetate buffer volume is twice the total volume of the lipid mixture, pH=7.2)”.

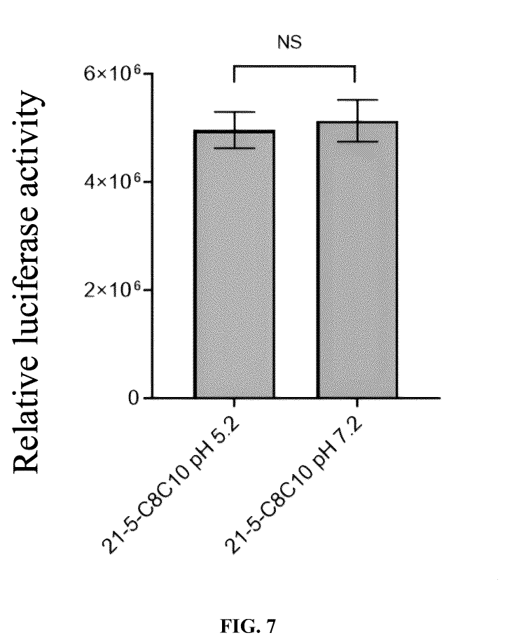

The result of relative luciferase activity is shown in FIG. 7. The results show that LNP can still load self-replicating mRNA normally under a buffer condition of pH=7.2, and can express the target protein efficiently. This shows that the ionizable lipid compound, that is, diethanolamine lipid, not only relies on electrostatic attraction to load RNA, but also relies on hydrogen bonding and van der Waals force interaction under neutral conditions to adsorb self-replicating mRNA onto the surface of LNP.

### Embodiment 6

This embodiment verifies the efficiency of ionizable lipid delivery of self-replicating mRNA encoding firefly luciferase (Luc) (saRNA-Luc), modified mRNA encoding firefly luciferase (Luc) (modRNA-Luc), and circular mRNA encoding firefly luciferase (Luc) (circRNA-Luc) in a 293T cell line. Ionizable lipids 21-5-C8, 22-5-C8, 21-6-C8, 22-6-C8, 21-5-C6C8, 22-5-C6C8, 21-6-C6C8, 21-6-C6C8, 21-5-C8C10, 22-5-C8C10, 21-6-C8C10, 21-6-C8C10 and commercial lipid ALC-0315 are used to deliver saRNA, modRNA and circRNA, respectively.

Specific steps are as follows:

### 1. Cell Culture

One day prior to the experiment, the cultured 293T cells are seeded in a 96-well cell culture plate (at a density of 50%). The cell transfection experiments are performed when the cell density reaches approximately 70-80%.

### 2. Preparation of Lipid Nanoparticles LNP-saRNA-Luc, LNP-modRNA-Luc, and LNP-circRNA-Luc for Cell Transfection.

The following steps take LNP-saRNA-Luc as an example.

Ionizable lipids 21-5-C8, 22-5-C8, 21-6-C8, 22-6-C8, 21-5-C6C8, 22-5-C6C8, 21-6-C6C8, 21-6-C6C8, 21-5-C8C10, 22-5-C8C10, 21-6-C8C10, 21-6-C8C10 are dissolved with cholesterol, and dimyristoyl glycerol-polyethylene glycol 2000 (PEG-DMG2000) in anhydrous ethanol at specified concentrations. The components are uniformly mixed at a molar ratio of ionizable lipid:Cholesterol:PEG-DMG2000=30:48:2.5. Concurrently, 150 ng saRNA-Luc is dissolved in sodium acetate buffer (the volume of sodium acetate buffer is twice the total lipid mixture volume, pH=7.2). Then, the saRNA buffer solution is rapidly mixed with the lipid mixture and incubated at room temperature for 20 min to allow assembly into stable LNPs. The LNP formulation is diluted with two volumes of sterile PBS and added to a 96-well cell culture plate for transfection. The optimal N/P ratio of ionizable lipid to saRNA is 18:1, which represents the molar ratio between the protonated amino groups of the lipid and the phosphate groups on the saRNA.

Positive control group: commercial lipid ALC-0315 assemblies LNPs according to its public preparation method.

Negative control group: 293T cells are cultured normally without transfection.

### 3. Cell Transfection Efficiency Analysis

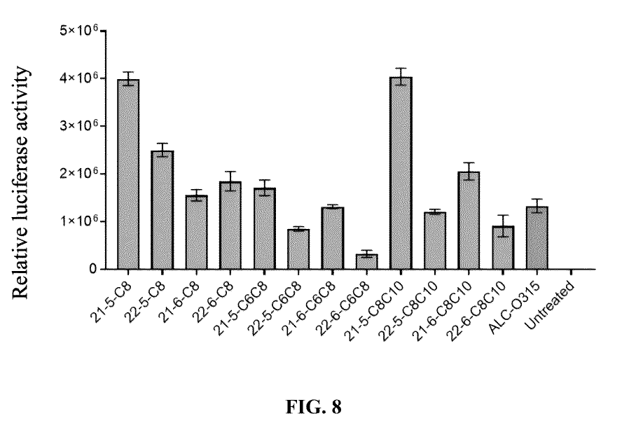

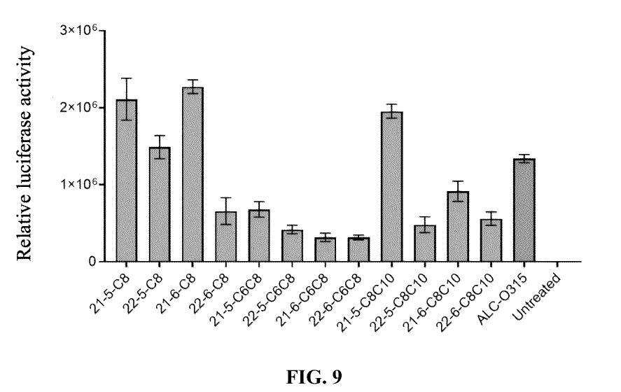

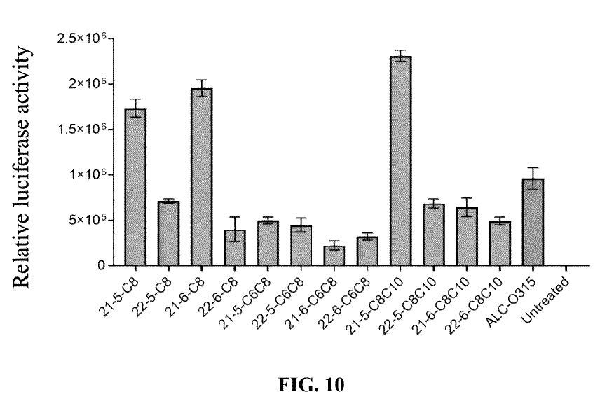

After 36 hours of cell transfection, the medium in the 96-well cell culture plate is aspirated. A cell lysis buffer is added to lyse the cells on ice for 25 min. Following centrifugation, the supernatant is collected and transferred to a white 96-well detecting plate. Then the firefly luciferase substrate is added, and the firefly luciferase activity (chemiluminescence) is measured using a microplate reader. The results of relative luciferase activity are shown in FIG. 8 (LNP-saRNA), FIG. 9 (LNP-modRNA), and FIG. 10 (LNP-circRNA). The results show that the ionizable lipids of the present disclosure significantly enhance the transfection efficiency of self-replicating mRNA, modified mRNA, and circular RNA, enabling efficient delivery of all three RNA types. Specifically, when the ionizable lipid headgroup is 21, which exhibits the highest RNA expression efficiency. Representative lipids 21-5-C8, 21-6-C8, and 21-5-C8C10, exhibit extremely high transfection efficiency across all three RNAs, exceeding the performance of the commercial lipid ALC-0315 by approximately 2 to 3 fold. This indicates that the ionizable lipid compound of the present disclosure, namely diethanolamine lipid, can efficiently deliver self-replicating mRNA, modRNA and circRNA in neutral buffer under three-component conditions.

### Embodiment 7

In this embodiment, the ionizable lipid compound 21-5-C8C10 is used to deliver a self-replicating mRNA encoding firefly luciferase (Luc) (saRNA-Luc) in Balb/c6 mice. The formulation is administered via intramuscular injection. Then the expression is detected in IVIS at 2, 5, 6, 8, 10, 12, 15, and 20 days post-injection.

Specific steps are as follows:

The experimental groups are performed as follows: 21-5-C8C10 (pH=7.2) and 21-5-C8C10 (pH=5.2). The ionizable lipid, cholesterol, and dimyristoyl glycerol-polyethylene glycol 2000 (PEG-DMG2000) are dissolved together in anhydrous ethanol at specified concentrations. Concurrently, 1.5 g saRNA-Luc is dissolved in sodium acetate buffer (the volume of sodium acetate buffer is twice the total lipid mixture volume, pH=7.2 or 5.2). This saRNA-Luc dissolved in the buffer solution is added to the lipid mixture solution and rapidly mixed uniformly to assemble into LNPs. The mixture is incubated at room temperature for 10 min. Subsequently, the solution is dialyzed for 1 h in PBS using a dialysis bag (14000 MW), and is administered via intramuscular injection to mice (n=4 per group). The molar ratio of ionizable lipid compound 21-5-C8C10:cholesterol:DMG-PEG2000 of 30:48:2.5 is employed. The RNA is pre-mixed in sodium acetate buffer, and the resulting nanoparticles possess an N/P ratio of 18:1.

### In Vivo Imaging Results Analysis

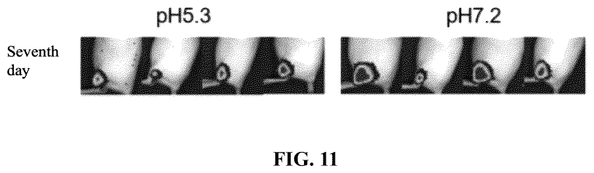

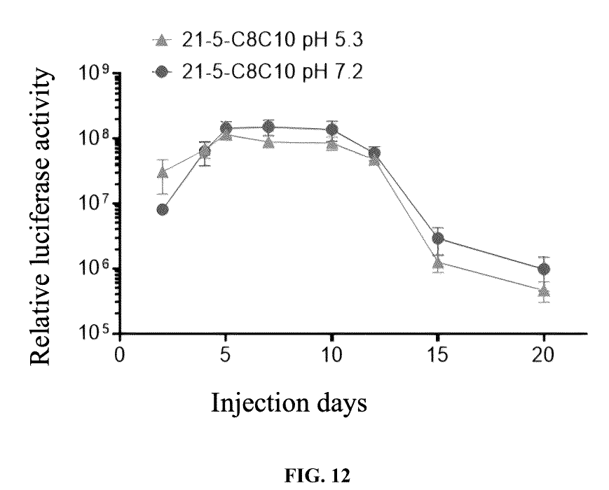

The results of IVIS (FIG. 11, and FIG. 12) show that all lipid nanoparticles are successfully expressed on the second day after intramuscular injection of saRNA-Luc. The expression values progressively increased over time. The expression values for self-replicating mRNA peaked between 7 to 10 days post-injection, while the expression value of modRNA peaked at approximately 48 h. This indicates that ionizable lipid-delivered self-replicating mRNA exhibits both a longer expression duration and a higher expression level, which is anticipated to contribute to a more sustained immune effect in subsequent mRNA vaccine applications.

### Embodiment 8

In this embodiment, the ionizable lipid compound 21-5-C8C10 and the commercial lipid ALC-0315 are used to deliver a self-replicating mRNA encoding firefly luciferase (Luc) (saRNA-Luc) in Balb/c6 mice. The formulation is administered via intramuscular injection. Then the expression is detected in IVIS at 2, 5, 7, 10, and 14 days post-injection.

Specific steps are as follows:

The experimental groups are performed as follows: four different components of 21-5-C8C10 (named A, B, C, and D). Group A is 21-5-C8C10:Cholesterol:DMG-PEG2000=30:48:1; group B is 21-5-C8C10:Cholesterol:DMG-PEG2000=30:48:2; group C is 21-5-C8C10:Cholesterol:DMG-PEG2000=30:48:2.5; and group D is 21-5-C8C10:Cholesterol:DMG-PEG2000=30:48:5. The ionizable lipid, cholesterol, and dimyristoyl glycerol-polyethylene glycol 2000 (PEG-DMG2000) are dissolved together in anhydrous ethanol at specified concentrations. Concurrently, 1.5 μg saRNA-Luc is dissolved in sodium acetate buffer (the volume of sodium acetate buffer is twice the total lipid mixture volume, pH=7.2). This saRNA-Luc dissolved in the buffer solution is added to the lipid mixture solution and rapidly mixed uniformly to assemble into LNPs. The mixture is incubated at room temperature for 10 min. Subsequently, the solution is dialyzed for 1 h in PBS using a dialysis bag (14000 MW), and is administered via intramuscular injection to mice (n=4 per group). The RNA is pre-mixed in sodium acetate buffer, and the resulting nanoparticles possess an N/P ratio of 18:1.

Positive control group: commercial lipid ALC-0315 assemblies LNPs according to its public preparation method.

### In Vivo Imaging Results Analysis

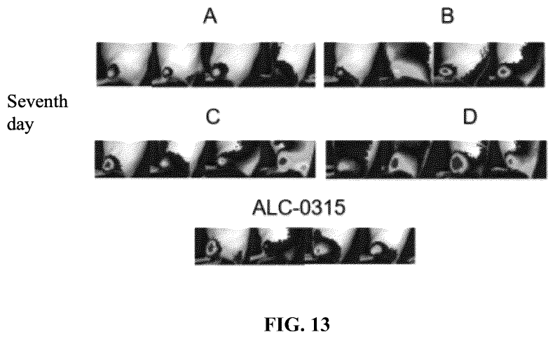

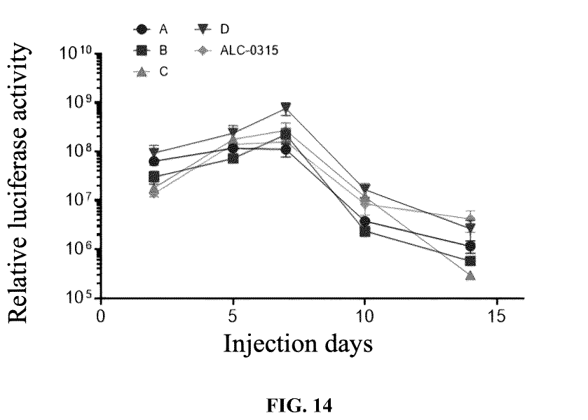

The results of IVIS (FIG. 13, and FIG. 14) show that the composition of the LNP formulation significantly influences the in vivo expression efficiency of self-replicating mRNA. Expression is successfully detected for all LNP formulations after intramuscular injection of saRNA-Luc. The self-replicating mRNA expression level peaked at 7 days post-injection. Notably, the delivery efficiency of the 21-5-C8C10 experimental group is better than that of ALC-0315, achieving a peak expression value approximately fivefold higher.

The foregoing embodiments are preferred embodiments of the present disclosure, and the scope of the present disclosure is not limited to these embodiments. Any modifications, alterations, substitutions, combinations, or simplifications made without departing from the spirit and principles of the present disclosure shall be considered equivalent substitutions and all fall within the scope of protection of the present disclosure.

What is claimed is:

1. An ionizable lipid compound comprising structural formulas of the ionizable lipid compounds of Formula I or Formula II, or their stereoisomers, their tautomers, or their pharmaceutically acceptable salts: 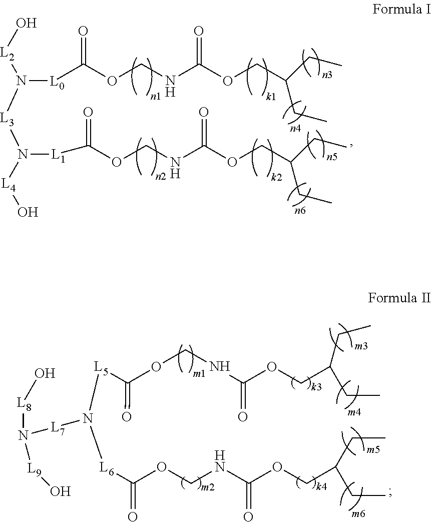
wherein L₀, L₁, L₂, L₃, L₄, L₅, L₆, L₇, L₈, and L₉ are each independently selected from C₁-C₈ alkyl;
n₁, n₂, n₃, n₄, n₅, n₆, m₁, m₂, m₃, m₄, m₅, and m₆ are all natural numbers, and each is independently selected from the natural numbers of 1-20;
k₁, k₂, k₃ and k₄ are all natural numbers, and each is independently selected from the natural numbers of 0-4.

2. The ionizable lipid compound according to claim 1, wherein the compound of Formula I has a structure selected from Formula I-1 or Formula I-2:

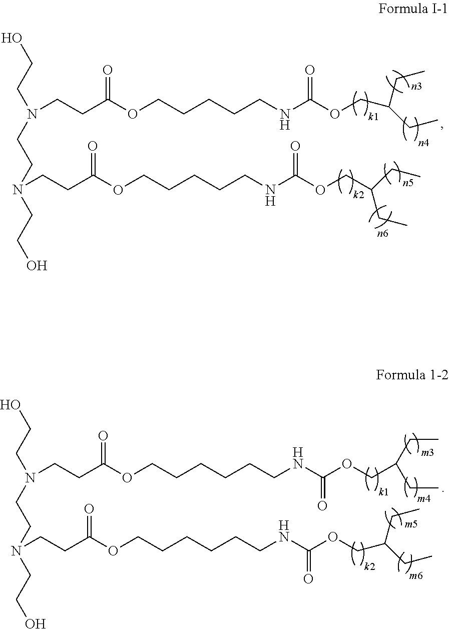

3. The ionizable lipid compound according to claim 1, wherein the compound of Formula II has a structure selected from Formula II-1 or Formula II-2:

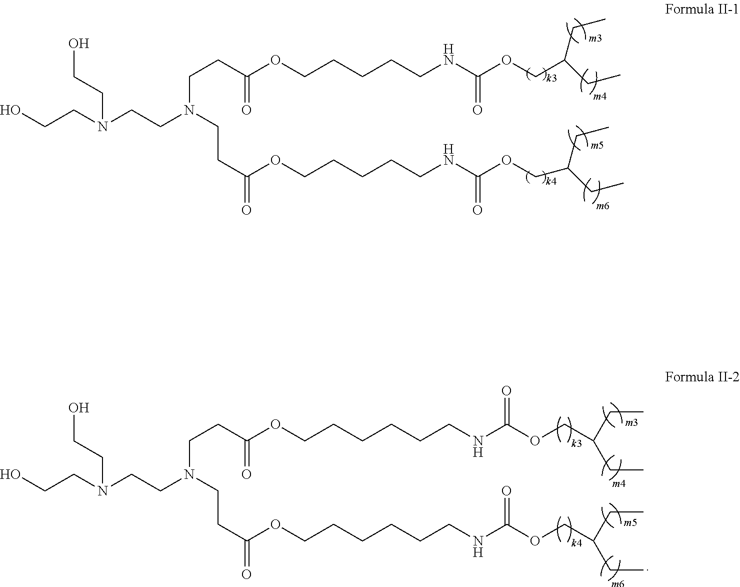

4. The ionizable lipid compound according to claim 1, wherein the ionizable lipid compound is selected from the following compounds:

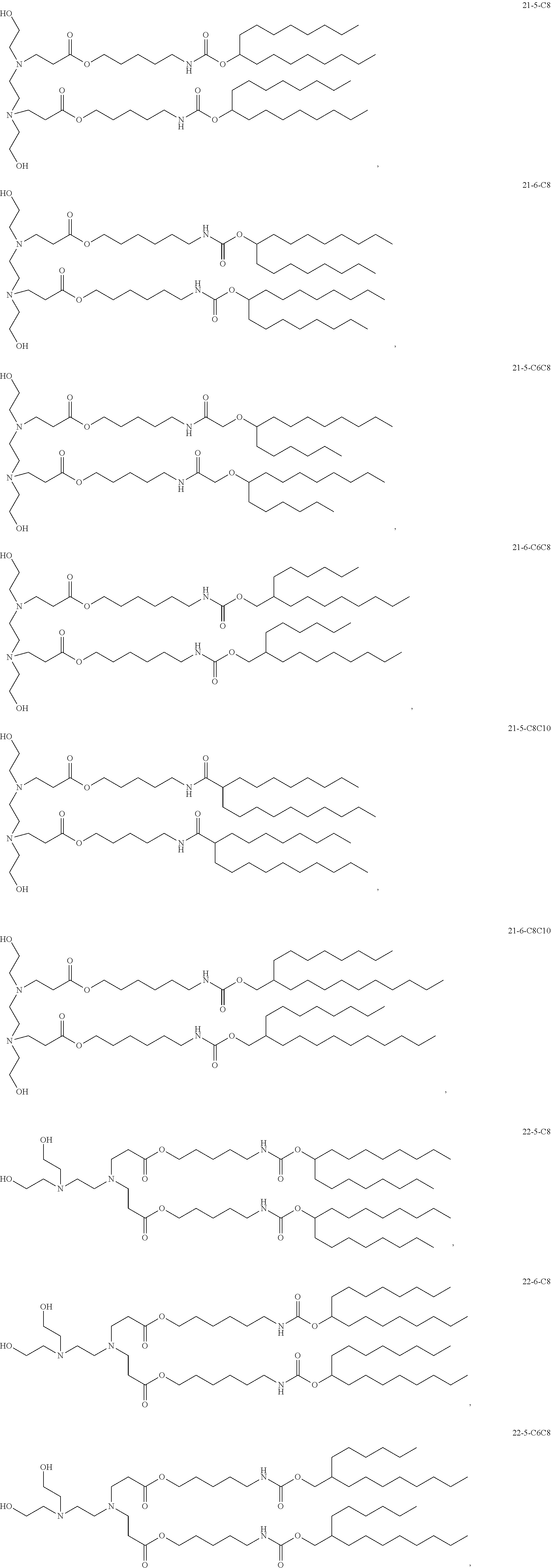

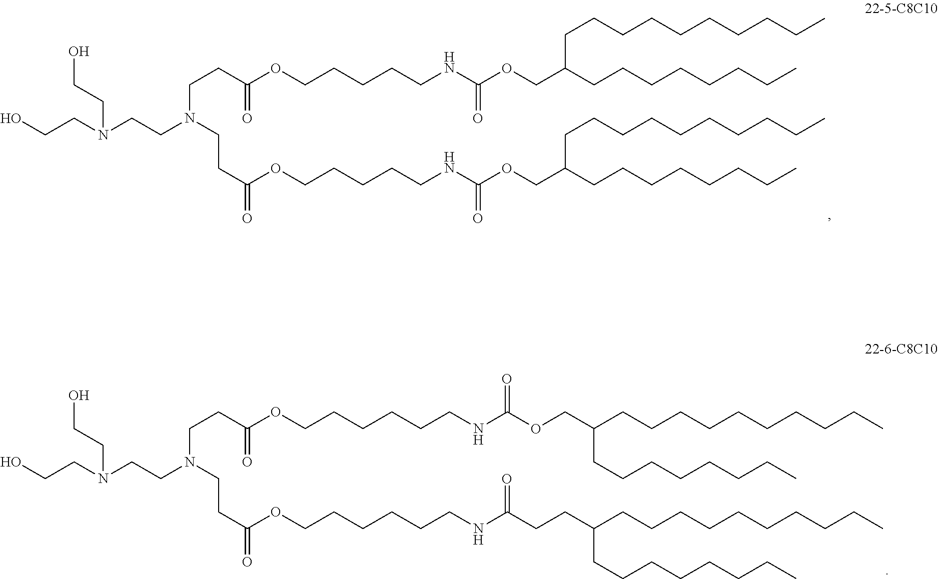 [SMILES: CCCCCCCCCCC(CCCCCCCC)COC(=O)NCCCCCOC(=O)CCN(CCC(=O)OCCCCCNC(=O)OCC(CCCCCCCC)CCCCCCCCCC)CCN(CCO)CCO ; CCCCCCCCCCC(CCCCCCCC)CCC(=O)NCCCCCCOC(=O)CCN(CCC(=O)OCCCCCCNC(=O)OCC(CCCCCCCC)CCCCCCCCCC)CCN(CCO)CCO]

5. A method for preparing the ionizable lipid compound according to claim 1, comprising the following steps:
reacting a compound of Formula 1 with N,N′-carbonyldiimidazole, followed by reaction with a compound of Formula 3, and subsequently reacting with a C1-C8 alkenyl acyl chloride, to yield an intermediate A; reacting the intermediate A with a compound of Formula 6 to yield the compound of Formula I; and
reacting the intermediate A with a compound of Formula 7 to yield the compound of Formula II;

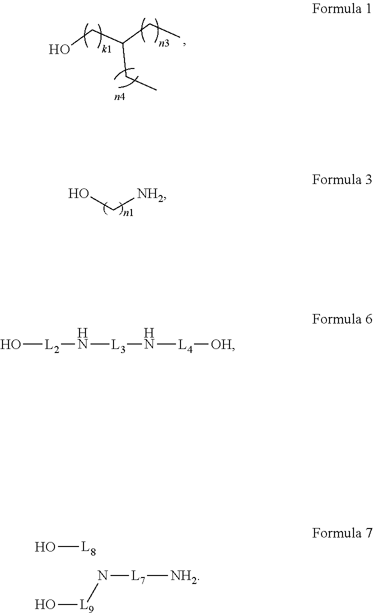

6. A lipid nanoparticle, comprising the ionizable lipid compound according to claim 1.

7. The lipid nanoparticle according to claim 6, wherein the lipid nanoparticle further comprises at least one of the structural lipids, auxiliary lipids, polyethylene glycol-modified lipids, and polymers.

8. The lipid nanoparticle according to claim 7, wherein the lipid nanoparticles contain 20%-65% ionizable lipid compounds, 0%-40% auxiliary lipids, 20%-60% structural lipids, and 0.1%-10% polyethylene glycol-modified lipids.

9. A drug composition, comprising the lipid nanoparticle according to claim 6.

10. The drug composition according to claim 9, wherein the drug composition further comprises drug active ingredients and/or pharmaceutically acceptable excipients, preferably, the drug active ingredient comprises at least one of a nucleic acid, a small molecule, or a protein drug.
